(19) United States
(12) Patent Application Publication
(10) Pub. No.: US 2011/0027829 A1
(43) Pub. Date: Feb. 3, 2011

(54) Methods and Compositions

(75) Inventors: Heinz Neumann, Cambridge (GB); Jason Chin, Cambridge (GB)

Correspondence Address: MCANDREWS HELD & MALLOY, LTD, 500 WEST MADISON STREET, SUITE 3400, CHICAGO, IL, 60661, US

(73) Assignee: MEDICAL RESEARCH COUNCIL, London (GB)

(21) Appl. No.: 12/739,247

(22) PCT Filed: Oct. 27, 2008
(86) PCT No.: PCT/GB2008/003611

§ 371 Date: Apr. 22, 2010

(30) Foreign Application Priority Data
Oct. 30, 2007 (GB) 0721291.3

(51) Int. Cl. C12P 21/00; C12N 9/00; C07H 21/00; C07K 2/00; C07K 14/00; C12N 15/63; C12N 1/00

(52) U.S. Cl. 435/68.1; 435/183; 536/23.2; 530/300; 530/350; 435/320.1; 435/243

(57) ABSTRACT

The invention relates to a tRNA synthetase capable of binding N^{⊂}-acetyl lysine, in particular, the invention relates to a tRNA synthetase capable of binding N^{∈}-acetyl lysine wherein said synthetase comprises a polypeptide having at least 90% sequence identity to the amino acid sequence of MbPy1RS. The invention also relates to a method of making a polypeptide comprising N^{∈}-acetyl lysine comprising arranging for the translation of a RNA encoding said polypeptide, wherein said RNA comprises an amber codon, wherein said translation is carried out in the presence of a polypeptide according to any of claims 1 to 11 and in the presence of tRNA capable of being charged with N^{∈}-acetyl lysine, and in the presence of N^{∈}-acetyl lysine.

### FIELD OF THE INVENTION

The invention is in the field of production of biologically important macromolecules which are acetylated. In particular, the invention is in the field of incorporation of N^{ε}-acetyl-lysine into polypeptides.

### BACKGROUND TO THE INVENTION

The genetic code of prokaryotic and eukaryotic organisms has been expanded to allow the in vivo, site-specific incorporation of over 20 designer unnatural amino acids in response to the amber stop codon. This synthetic genetic code expansion is accomplished by endowing organisms with evolved orthogonal aminoacyl-tRNA synthetase/tRNA_{CUA} pairs that direct the site-specific incorporation of an unnatural amino acid in response to an amber codon. The orthogonal aminoacyl-tRNA synthetase aminoacylates a cognate orthogonal tRNA, but no other cellular tRNAs, with an unnatural amino acid, and the orthogonal tRNA is a substrate for the orthogonal synthetase but is not substantially aminoacylated by any endogenous aminoacyl-tRNA synthetase.

Genetic code expansion in E. coli using evolved variants of the orthogonal Methanococcus jannaschii tyrosyl-tRNA synthetase/tRNA_{CUA} pair greatly increases unnatural amino acid-containing protein yield since, in contrast to methods that rely on the addition of stoichiometrically pre-aminoacylated suppressor tRNAs to cells or to in vitro translation reactions, the orthogonal tRNA_{CUA} is catalytically re-acylated by its cognate aminoacyl-tRNA synthetase enzyme, thus aminoacylation need not limit translational efficiency.

Many potential applications of unnatural amino acid mutagenesis, including the translational incorporation of amino acids corresponding to post-translational modifications present at multiple sites in proteins such as acetylation, require more efficient methods of incorporation to make useful amounts of protein. Moreover the introduction of biophysical probes and chemically precise perturbations into proteins in their native cellular context offers the exciting possibility of understanding and controlling cellular functions in ways not previously possible.

N^{ε}-acetylation of lysine is a reversible post-translational modification with a regulatory role to rival phosphorylation in eukaryotic cells¹⁻¹⁴. No general methods to synthesize proteins containing N^{ε}-acetyl-lysine at defined sites exist.

N^{ε}-acetylation of lysine was first described on histones²¹. Lysine acetylation and de-acetylation are mediated by histone acetyl transferases (HATs) and histone deacetylases (HDACs) respectively. In recent years it has emerged that hundreds of eukaryotic proteins (beyond histones) are regulated by acetylation, including more than 20% of mitochondrial proteins²⁰.

Despite the huge importance of lysine acetylation there is no general method of producing homogeneous recombinant proteins that contain N^{ε}-acetyl-lysine at defined sites. Semi-synthetic methods to install N^{ε}-acetyl-lysine using native chemical ligation were employed in demonstrating the role of H4 K16 in chromatin decompaction¹. These studies give a taste of the impact that a general method to produce homogeneously acetylated proteins would have on our understanding of the molecular role of acetylation in biology.

Current chemical based methods of acetylation require the synthesis of large quantities of modified peptide thioester, which is a drawback. Furthermore, such known methods suffer from limitation to N-terminal residues.

Some researchers have used purified HAT complexes to acetylate recombinant proteins. However this is often an unsatisfactory solution because: i) the HATs for a particular modifications may be unknown; ii) tour-de-force efforts are often required to prepare active HAT complexes; iii) HAT mediated reactions are often difficult to drive to completion leading to a heterogeneous sample; and iv) HATs may acetylate several sites, making it difficult to interrogate the molecular consequences of acetylation at any one site.

The present invention seeks to overcome problem(s) associated with the prior art.

### SUMMARY OF THE INVENTION

N^{∈}-acetylation of lysine is a post translational modification of substantial biological importance. The study of N^{∈}-acetylation in the prior art is extremely difficult. Prior art techniques for producing N^{∈}-acetylated proteins have relied on chemical or semi-synthetic methods of installing N^{∈}-acetyl lysine into the polypeptides of interest. Some of these processes are extremely technically demanding, whilst others have severe limitations such as restriction to modification of N-terminal residues. No general method of producing homogeneous recombinant proteins comprising N^{∈}-acetyl lysine is known in the prior art.

The present inventors have devised a way of exploiting the naturally occurring polypeptide synthesis machinery (translational machinery) of the cell in order to reliably incorporate N^{∈}-acetyl lysine into polypeptides at precisely defined locations. Specifically, the inventors have developed a tRNA synthetase which has been modified to accept N^{∈}-acetyl lysine and to catalyse its incorporation into transfer RNA (tRNA). Thus, the present inventors have produced a new enzymatic activity which is previously unknown in nature. Furthermore, the inventors have evolved this novel enzyme into a suitable tRNA synthetase/tRNA pairing which can be used in order to specifically incorporate N^{∈}-acetyl lysine into proteins at the point of synthesis and at position(s) chosen by the operator.

Thus, the present inventors provide for the first time a novel tRNA synthetase, and a corresponding new approach to the production of polypeptides incorporating N^{∈}-acetyl lysine. These new materials and techniques enable the production of homeogeneous samples of polypeptide which each comprise the desired post translational modification. This simply has not been possible using the existing chemistry based techniques known in the prior art.

The invention is based upon these remarkable findings.

Thus, in one aspect the invention provides a tRNA synthetase capable of binding N^{∈}-acetyl lysine.

In another aspect, the invention relates to a tRNA synthetase as described above wherein said synthetase comprises a polypeptide having at least 90% sequence identity to the amino acid sequence of MbPy1RS. Suitably said identity is assessed across at least 50 contiguous amino acids. Suitably said identity is assessed across a region comprising amino acids corresponding to L266 to C313 of MbPy1RS.

In another aspect, the invention relates to a tRNA synthetase as described above wherein said tRNA synthetase polypeptide comprises amino acid sequence corresponding to the amino acid sequence of at least L266 to C313 of MbPy1RS, or a sequence having at least 90% identity thereto.

Suitably said polypeptide comprises a mutation relative to the wild type MbPy1RS sequence at one or more of L266, L270, Y271, L274 or C313.

Suitably said at least one mutation (i.e. said one or more mutation(s)) is at L270, Y271, L274 or C313.

Suitably said at least one mutation is at L270, L274 or C313.

Suitably said tRNA synthetase comprises Y271L.

Suitably said tRNA synthetase comprises Y271F.

Suitably said tRNA synthetase comprises L266V.

Suitably said tRNA synthetase comprises L2701, Y271L, L274A, and C313F.

Suitably said tRNA synthetase comprises L266V, L2701, Y271F, L274A, and C313F.

In another aspect, the invention relates, to a nucleic acid comprising nucleotide sequence encoding a polypeptide as described above.

In another aspect, the invention relates to use of a polypeptide as described above to charge a tRNA with N^{∈}-acetyl lysine. Suitably said tRNA comprises MbtRNA_{CUA} (i.e. suitably said tRNA comprises the publicly available wild type Methanosarcina barkeri tRNACUA sequence as encoded by the MbPy1T gene.).

In another aspect, the invention relates to a method of making a polypeptide comprising N^{∈}-acetyl lysine comprising arranging for the translation of a RNA encoding said polypeptide, wherein said RNA comprises an amber codon, wherein said translation is carried out in the presence of a polypeptide as described above and in the presence of tRNA capable of being charged with N^{∈}-acetyl lysine, and in the presence of N^{∈}-acetyl lysine.

Suitably said translation is carried out in the presence of an inhibitor of deacetylation.

Suitably said inhibitor comprises nicotinamide (NAM).

In another aspect, the invention relates to a method of making a polypeptide comprising N^{∈}-acetyl lysine, said method comprising modifying a nucleic acid encoding said polypeptide to provide an amber codon at one or more position(s) corresponding to the position(s) in said polypeptide where it is desired to incorporate N^{∈}-acetyl lysine. Suitably modifying said nucleic acid comprises mutating a codon for lysine to an amber codon (TAG).

In another aspect, the invention relates to a homogeneous recombinant protein comprising N^{∈}-acetyl lysine. Prior art proteins have been heterogeneous with regard to their N^{∈}-acetyl lysine content. Suitably said protein is made by a method as described above.

In another aspect, the invention relates to a vector comprising nucleic acid as described above. Suitably said vector further comprises nucleic acid sequence encoding a tRNA substrate of said tRNA synthetase. Suitably said tRNA substrate is encoded by the MbPy1T gene (see above).

In another aspect, the invention relates to a cell comprising a nucleic acid as described above, or comprising a vector as described above.

In another aspect, the invention relates to a cell as described above which further comprises an inactivated de-acetylase gene. Suitably said deactivated de-acetylase gene comprises a deletion or disruption of CobB.

In another aspect, the invention relates to a kit comprising a vector as described above, or comprising a cell as described above, and an amount of nicotinamide.

In another aspect, the invention relates to a method of making a tRNA synthetase capable of binding N^{∈}-acetyl lysine, said method comprising mutating a nucleic acid encoding a parent tRNA synthetase sequence at one or more of L266, L270, Y271, L274 or C313, and selecting one or more mutants which are capable of binding N^{∈}-acetyl lysine.

### DETAILED DESCRIPTION OF THE INVENTION

To address the prior art deficit in methods to synthesize acetylated proteins we envisioned genetically encoding the incorporation of N^{ε}-acetyl-lysine into proteins with high translational fidelity and efficiency in response to the amber codon, via the generation of an orthogonal N^{ε}-acetyl-lysyl-tRNA synthetase/tRNA pair. Here we describe methods and materials for genetically incorporating N^{ε}-acetyl-lysine in response to the amber codon in Escherichia coli (E. coli), to produce site-specifically acetylated recombinant proteins. We further enable such proteins to be produced homogeneously, which has not been possible with prior art based techniques. We demonstrate that the Methanosarcina barkeri pyrrolysyl-tRNA synthetase (MbPy1RS)/MbtRNA_{CUA} pair¹⁵⁻¹⁹ is orthogonal in E. coli, and has a comparable efficiency to a previously reported useful pair. We evolve this pair for site-specific incorporation of N^{ε}-acetyl-lysine in response to the amber codon with high translational fidelity and efficiency. Furthermore, we successfully eradicate the initially observed post-translational deacetylation. These strategies find wide application in deciphering the role of acetylation in the epigenetic code proposed for chromatin modifications^{2, 3}, and in a broader understanding of the cellular role of N^{ε}-acetylation²⁰.

### DEFINITIONS

The term ‘comprises’ (comprise, comprising) should be understood to have its normal meaning in the art, i.e. that the stated feature or group of features is included, but that the term does not exclude any other stated feature or group of features from also being present.

Networks of molecular interactions in organisms have evolved through duplication of a progenitor gene followed by the acquisition of a novel function in the duplicated copy. Described herein are processes that artificially mimic the natural process to produce orthogonal molecules: that is, molecules that can process information in parallel with their progenitors without cross-talk between the progenitors and the duplicated molecules. Using these processes, it is now possible to tailor the evolutionary fates of a pair of duplicated molecules from amongst the many natural fates to give a predetermined relationship between the duplicated molecules and the progenitor molecules from which they are derived. This is exemplified herein by the generation of orthogonal tRNA synthetase-orthogonal tRNA pairs that can process information in parallel with wild-type tRNA synthetases and tRNAs but that do not engage in cross-talk between the wild-type and orthogonal molecules. In some embodiments the tRNA itself may retain its wild type sequence. In those embodiments, suitably said entity retaining its wild type sequence is used in a heterologous setting i.e. in a background or host cell different from its naturally occurring wild type host cell. In this way, the wild type entity may be orthogonal in a functional sense without needing to be structurally altered. Orthogonality and the accepted criteria for same are discussed in more detail below.

The Methanosarcina barkeri Py1S gene encodes the MbPy1RS tRNA synthetase protein. The Methanosarcina barkeri Py1T gene encodes the MbtRNA_{CUA} tRNA.

### Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods, are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension. Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

In the context of the present document, a homologous amino acid sequence is taken to include an amino acid sequence which is at least 15, 20, 25, 30, 40, 50, 60, 70, 80 or 90% identical, preferably at least 95 or 98% identical at the amino acid level. Suitably this identity is assessed over at least 50 or 100, preferably 200, 300, or even more amino acids with the relevant polypeptide sequence(s) disclosed herein, most suitably with the full length progenitor (parent) tRNA synthetase sequence. Suitably, homology should be considered with respect to one or more of those regions of the sequence known to be essential for protein function rather than non-essential neighbouring sequences. This is especially important when considering homologous sequences from distantly related organisms.

Most suitably sequence identity should be judged across at least the contiguous region from L266 to C313 of the amino acid sequence of MbPy1RS, or the corresponding region in an alternate tRNA synthetase.

The same considerations apply to nucleic acid nucleotide sequences, such as tRNA sequence(s).

### Reference Sequence

When particular amino acid residues are referred to using numeric addresses, the numbering is taken using MbPy1RS (Methanosarcina barkeri pyrrolysyl-tRNA synthetase) amino acid sequence as the reference sequence (i.e. as encoded by the publicly available wild type Methanosarcina barkeri Py1S gene). This is to be used as is well understood in the art to locate the residue of interest. This is not always a strict counting exercise—attention must be paid to the context. For example, if the protein of interest is of a slightly different length, then location of the correct residue in that sequence correseponding to (for example) Y271 may require the sequences to be aligned and the equivalent or corresponding residue picked, rather than simply taking the 271^{st} residue of the sequence of interest. This is well within the ambit of the skilled reader.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably a randomisation of said site is used, for example as described herein in connmection with the evolution and adaptation of tRNA synthetase of the invention. As a default mutation, alanine (A) may be used. Suitably the mutations used at particular site(s) are as set out herein.

A fragment is suitably at least 10 amino acids in length, suitably at least 25 amino acids, suitably at least 50 amino acids, suitably at least 100 amino acids, suitably at least 200 amino acids, suitably at least 250 amino acids, suitably at least 300 amino acids, suitably at least 313 amino acids, or suitably the majority of the tRNA synthetase polypeptide of interest.

### Polypeptides of the Invention

Suitably the polypeptide comprising N^{∈}-acetyl lysine is a nucleosome or a nucleosomal polypeptide.

Suitably the polypeptide comprising N^{∈}-acetyl lysine is a chromatin or a chromatin associated polypeptide.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected, into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals.

These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### Optimisation

Unnatural amino acid incorporation in in vitro translation reactions can be increased by using S30 extracts containing a thermally inactivated mutant of RF-1. Temperature sensitive mutants of RF-1 allow transient increases in global amber suppression in vivo. Increases in tRNA_{CUA} gene copy number and a transition from minimal to rich media may also provide improvement in the yield of proteins incorporating an unnatural amino acid in E. coli.

### Industrial Application

N^{ε}-acetylation regulates diverse cellular processes. The acetylation of lysine residues on several histones modulates chromatin condensation¹, may be an epigenetic mark as part of the histone code², and orchestrates the recruitment of factors involved in regulating transcription, DNA replication, DNA repair, recombination, and genome stability in ways that are beginning to be deciphered³. Over 60 transcription factors and co-activators are acetylated, including the tumor suppressor p53⁴, and the interactions between components of the transcription, DNA replication, DNA repair, and recombination machinery are regulated by acetylation^{5, 6}. Acetylation is important for regulating cytoskeletal dynamics, organizing the immunological synapse and stimulating kinesin transport^{7, 8}. Acetylation is also an important regulator of glucose, amino acid and energy metabolism, and the activity of several key enzymes including histone acetyl-transferases, histone deacetylases, acetyl CoA synthases, kinases, phosphatases, and the ubiquitin ligase murine double minute are directly regulated by acetylation⁹. Acetylation is a key regulator of chaperone function¹⁰, protein trafficking and folding¹¹, stat3 mediated signal transduction¹² and apoptosis¹³. Overall it is emerging that N^{ε}-acetylation is a modification with a diversity of roles and a functional importance that rivals phosphorylation¹⁴. Thus, there are clear utilities and industrial applications for the methods and materials disclosed herein, both in the production of saleable products and in facilitation of the study of essential biological processes as noted above.

### Further Applications

Inhibition of deacetylase may be by any suitable method known to those skilled in the art. Suitably inhibition is by gene deletion or disruption of endogenous deacetylase(s). Suitably such disrupted/deleted acetylase is CobB. Suitably inhibition is by inhibition of expression such as inhibition of translation of endogenous deacetylase(s). Suitably inhibition is by addition of exogenous inhibitor such as nicotinamide.

In one aspect the invention relates to the addition of N^{ε}-acetyl-lysine to the genetic code of organisms such as Escherichia coli.

The invention finds particular application in synthesis of nucleosomes and/or chromatin bearing N^{ε}-acetyl-lysine at defined sites on particular histones. One example of such an application is for determining the effect of defined modifications on nucleosome and chromatin structure and function^{1, 26}.

The MbPy1RS/MbtRNA_{CUA} pair may be further evolved for the genetic incorporation of mono-, di- and/or tri-methyl-lysine to explore the roles of these modifications on histone structure and function, and/or their role in an epigenetic code¹⁴. Moreover the methods described here may also be applied to genetically incorporate lysine residues derivatized with diverse functional groups and/or biophysical probes into proteins in E. coli.

Since MbPy1RS does not recognize the anticodon of MbtRNA_{CUA}¹⁸ it is further possible to combine evolved MbPy1RS/MbtRNA pairs with other evolved orthogonal aminoacyl-tRNA synthetase/tRNA_{CUA} pairs, and/or with orthogonal ribosomes with evolved decoding properties²⁷ to direct the efficient incorporation of multiple distinct useful unnatural amino acids in a single protein.

### BRIEF DESCRIPTION OF THE FIGURES

FIG. 1 shows a photograph and a graph which demonstrate that the MbPy1RS MbtRNA_{CUA} pair efficiently and specifically incorporates N^{ε}-cyclopentyloxycarbonyl-L-lysine (Cyc) in response to an amber stop codon in the gene for myoglobin. A. Production of myoglobin from Myo4TAG-Py1T depends on the presence of Cyc in the growth medium. Myoglobin expressed in the presence of MjTyrRS/MjtRNA_{CUA} (lane 1) or MbPy1RS/MbtRNA_{CUA} in the presence or absence of 1 mM Cyc (lanes 2 and 3) was purified by Ni²⁺-affinity chromatography, analyzed by SDS-PAGE and stained with Coomassie. B. ESI-MS analysis of myoglobin produced by MjTyrRS/MjtRNA_{CUA} (Tyr) revealed a mass of 18433.2 Da (predicted 18431.2 Da) while the myoglobin produced by MbPy1RS/MbtRNA_{CUA} (Cyc) has a mass of 18510.7 Da. The expected mass difference (m(Cyc)−m(Tyr)=258.3 Da−181.2 Da=77.1 Da) corresponds well to the mass difference observed (77.5 Da).

FIG. 2 shows molecular structures which illustrate the design of an MbPy1RS for the genetic incorporation of N^{ε}-acetyl-lysine. A. Structure of lysine (1), pyrrolysine (2), and N^{ε}-acetyl-lysine (3). B. Structure of the active site of MbPy1RS bound to pyrrolysine. Amino acid residues that form the hydrophobic binding pocket of pyrrolysine, and are mutated in the library to each of the common 20 amino acids, are shown. The image was created using PyMol v0.99 (www.pymol.org) and PDB ID 2Q7H.

FIG. 3 shows photomicrographs and graphs illustrating that the evolved aminoacyl-tRNA synthetase efficiently incorporates N^{ε}-acetyl-lysine into proteins in response to an amber codon. A. Myoglobin produced in the presence of MjTyrRS/MjtRNA_{CUA} (lane 1) or AcKRS-2 in the absence or presence of 1 mM N^{ε}-acetyl-lysine (AcK, lanes 2 and 3) or in the presence of 1 mM acetyl-lysine and 50 mM nicotinamide (NAM, lane 4). Proteins were purified by Ni²⁺-affinity chromatography, separated by SDS-PAGE and either stained with Coomassie or transferred to nitrocellulose and detected with antibodies to the hexahistidine tag or acetyl-lysine. B. ESI-MS analysis of the purified acetylated myoglobin. Myoglobin expressed in the absence of nicotinamide (green,—NAM) produced two peaks of masses 18397.6 (b) and 18439.2 Da (a) which correspond to deacetylated and acetylated myoglobin (predicted masses: 18396.2 and 18438.2 Da). When myoglobin was expressed in the presence of 50 mM nicotinamide (blue,+NAM) only the peak for the acetylated protein was observed (c).

The invention is now described by way of example. These examples are intended to be illustrative, and are not intended to limit the appended claims.

### Examples

### Overview

Certain methanogenic bacteria, including Methanosarcina barkeri (Mb), incorporate pyrrolysine in response to the UAG codons present in several methyl-transferase genes^{15, 16}. The incorporation of pyrrolysine in Methanosarcina barkeri is directed by a pyrrolysyl-tRNA synthetase (MbPy1RS) and its cognate amber suppressor, MbtRNA_{CUA}, in response to an amber codon¹⁷. The MbPy1RS/MbtRNA_{CUA} pair functions in E. coli and MbtRNA_{CUA} is not an efficient substrate for endogenous aminoacyl-tRNA synthetases in E. coli^{16, 18}. The MbPy1RS/MbtRNA_{CUA} therefore appears to satisfy two of the three criteria for orthogonality with respect to endogenous aminoacyl-tRNA synthetases and tRNAs²².

These observations; in combination with the insight that acetyl-lysine is a sub-structure of pyrrolysine, led us to investigate the evolution of the MbPy1RS/MbtRNA_{CUA} into an N^{ε}-acetyl-lysyl-tRNA synthetase/tRNA_{CUA} pair for the genetic incorporation of acetyl-lysine into proteins expressed in E. coli.

### Examples

### General Methods

### Construction of Plasmids

Plasmid pMyo4TAG-Py1T encodes a myoglobin gene, with codon 4 replaced by an amber codon, under the control of an arabinose promoter. It also contains the Py1T gene with an lpp promoter and rrnC terminator. pMyo4TAG-Py1T was generated by the ligation of two PCR products. One PCR product was generated using pBADJYAMB4TAG²² as template in a PCR reaction that amplified the entire vector except the MjtRNA_{CUA} gene. This PCR used the primers pMyoNotF (5′-CAA GCG GCC GCG AAT TCA GCG TTA CAA GTA TTA CA-3′) and pMyoPstR (5′-GAC CAC TGC AGA TCC TTA GCG AAA GCT-3′). The second PCR product was generated by amplifying the Py1T gene from pREP-Py1T using primers PYLTPST13 (5′-GCG ACG CTG CAG TGG CGG AAA CCC CGG GAA TC-3′) and PYLTNOT15 (5′-GGA AAC CGC GCG GCC GCG GGA ACC TGA TCA TGT AGA TCG-3′). The two PCR products were digested with NotI and PstI and ligated with T4 DNA ligase to form pMyo4TAG-Py1T.

pREP-Py1T was derived from pREP(2) YC-JYCUA^{22, 28}. The MjtRNA_{CUA} gene in pREP(2) YC-JYCUA was deleted by Quickchange mutagenesis (Stratagene) creating unique BglII and SpeI sites downstream of the lpp promoter. This was performed using primers pREPDtf (5′-CTAGATCTATGACTAGTATCCTTAGCGAAAGCTAA-3′) and pREPDtr (5′-ATACTAGTCATAGATCTAGCGTTACAAGTATTACA-3′). The Py1T gene was made by PCR from primers pylTbegf (5′-GCT AGA TCT GGG AAC CTG ATC ATG TAG ATC GAA TGG ACT CTA AAT CCG TTC AGC C-3′ and py1Tendr (5′-GAT ACT AGT TGG CGG AAA CCC CGG GAA TCT AAC CCG GCT GAA CGG ATT TAG AGT C-3′) and cloned between BglII and SpeI in the intermediate vector.

pBAR-Py1T (which contains a toxic barnase gene with amber codons at positions Q2 and D44 under the control of an arabinose promoter and Py1T on an lpp promoter) was derived from pYOBB2 using the same strategy and primers used to create pREP-Py1T from pREP(2) YC-JYCUA.

### Library Construction

An E. coli codon optimized version of the MbPy1S gene was synthesized (Geneart). This ORF was cloned between the NdeI and PstI sites of pBK-JYRS²² replacing the MjTyrRS gene and producing pBK-Py1S. Three rounds of inverse PCR were performed on this template to randomize codons of L266, L270, Y271, L274, C313 and. W383, with the product of one round acting as a template for the next round. The following primers were used in each round of PCR reactions: (round 1) Py1SC313f (5′-GCG CAG GAA AGG TCT CAA ACT TTN NKC AAA TGG GCA GCG GCT GCA CCC GTG AAA AC-3′) and Py1SC313r (5′-GCG CAG AGT AGG TCT CAA GTT AAC CAT GGT GAA TTC TTC CAG GTG TTC TTT G-3′); (round 2) Py1SL266f (5′-GCG CAG GTC TCA CCG ATG NNK GCC CCG ACC NNK NNK AAC TAT NNK CGT AAA CTG GAT CGT ATT CTG CCG GGT C-3′) and Py1SL266r (5′-GCG CAG AGT AGG TCT CAT CGG ACG CAG GCA CAG GTT TTT ATC CAC GCG GAA AAT TTG-3′); (round 3) Py1SW383f2 (5′-GCG CAG GAA AGG TCT CAA AAC CGN NKA TTG GCG CGG GTT TTG GCC TGG AAC GTC TGC TG-3′) and Py1SW383r2 (5′-GCG CAG AGT AGG TCT CAG TTT ATC AAT GCC CCA TTC ACG ATC CAG GCT AAC CGG AC-3′). The enzymatic inverse PCR reactions were prepared in 100 μL aliquots containing 1× PCR buffer with MgCl₂(Roche), 200 μM of each dNTP, 0.8 μM of each primer, 100 ng template and 7 U Expand High Fidelity Polymerase (Roche). PCR reactions were run in 50 μl aliquots using the following temperature program: 2 min at 95° C., 9×(20 sec at 95° C., 20 sec at 65° C. [−1° C./cycle], 4 min at 68° C.), 31×(20 sec at 95° C., 20 sec at 58° C., 4 min at 68° C.), 9 min at 68° C.

The purified PCR reactions were digested, with DpnI and BsaI, ligated, precipitated and used to transform electrocompetent DH10B cells, as previously described²⁹. To increase the number of independent transformants after the last round of enzymatic inverse PCR the precipitated ligation product was amplified with Phi29 DNA polymerase in a 500 μl reaction, as previously described³⁰. The final transformation yielded a library of approximately 10⁸ mutants. The quality of the library was verified by sequencing twelve randomly chosen clones, which showed no bias in the nucleotides incorporated at the randomized sites.

Selection of N^{ε}-acetyl-lysine Specific Aminoacyl-tRNA Synthetases

E. coli DH10B harbouring the plasmid pREP-Py1T were transformed with the library of mutant synthetase clones, yielding 10⁹ transformants. Cells were incubated (16 h, 37° C., 250 r.p.m.) in 100 mL LB, supplemented with 12.5 μg ml⁻¹ tetracycline and 25 μg ml⁻¹ kanamycin (LB-KT). 2 mL of this culture was diluted 1:50 into fresh LB-KT containing 1 mM N^{ε}-acetyl-lysine (Bachem) and incubated (3-4 h, 37° C., 250 r.p.m.). 0.5 ml of the culture was plated onto LB-KT plates (24 cm×24 cm) supplemented with 1 mM acetyl-lysine and 50 μg ml⁻¹ chloramphenicol. After incubation (48 h, 37° C.) the plates were stripped of cells and plasmids isolated. The synthetase plasmids were ,resolved from the reporter plasmid by agarose gel electrophoresis and extracted using the Qiagen gel purification kit.

To select against synthetases that direct incorporation of natural amino acids in response to the amber codon plasmids isolated in this positive selection were used to transform DH10B containing plasmid pBar-Py1T. After electroporation the cells were recovered (3 h, 37° C., 250 r.p.m.) in SOB medium. Approximately 10⁷ cells were plated onto LB-agar plates (24 cm×24 cm) supplemented with 0.2% arabinose, 25 μg ml⁻¹ kanamycin and 25 μg m⁻¹ chloramphenicol. The plates were incubated for 24 h at 37° C. Cells from the resulting colonies were harvested and the synthetase plasmids isolated as described above.

The third round of selection was performed in the same way as the first, except that instead of harvesting the pool of synthetase plasmids we picked individual colonies and grew these in parallel in lmL of LB-KT. After overnight growth 200 μL of each culture was diluted 1:10 into fresh LB-KT and divided to give two identical 1 mL cultures derived from a single colony. One culture received 1 mM N^{ε}-acetyl-lysine and the other did not. After incubation (5 h, 37° C., 250 r.p.m.) the cells were pronged onto LB-KT plates with or without 1 mM N^{ε}-acetyl-lysine and containing increasing concentrations of chloramphenicol. Total plasmid DNA was isolated from 24 clones that showed strong N^{ε}-acetyl-lysine dependent chloramphenicol resistance. This DNA was digested with HindIII (which does not digest pBK-Py1S, but does dige20t pREP-Py1T) and used to transform DH10B. To confirm that the observed phenotypes did not result from mutations in the cells genome or mutations in the reporter plasmid cells containing pREP-Py1T were transformed with the isolated pBK-Py1S plasmids and tested for their ability to grow on increasing concentrations of chloramphenicol in the presence or absence of 2 mM N^{ε}-acetyl-lysine. Additionally, we analysed for the expression of GFP by scanning plates without chloramphenicol on a Storm Phosphoimager (Molecular Dynamics).

### Expression and Purification of Myoglobin Via Amber Suppression

E. coli DH10B was transformed with pBKPy1S, AcKRS-1 or AcKRS-2 and pMyo4TAG-Py1T. The cells were incubated (16 h, 37° C., 250 r.p.m.) in LB-KT. 1 liter of LB KT supplemented with 1 mM N^{ε}-acetyl-lysine or Cyc (Sigma) was inoculated with 50 mL of this overnight culture. After 2 h at 37° C. the culture was supplemented with 50 mM nicotinamide (Sigma) and grown for another 30 min. Protein expression was induced by addition of 0.2% arabinose. After a further 3 h cells were harvested and washed with PBS. Proteins were extracted by shaking at 25° C. in 30 mL BugBuster (Novagen) supplemented with protease inhibitor cocktail (Roche), 1 mM PMSF, 50 mM nicotinamide and approximately 1 mg ml⁻¹ lysozyme. The extract was clarified by centrifugation (15 min, 2500 g, 4° C.) and supplemented with 20 mM imidazole, and 50 mM Tris (pH 8.0) to give a total volume of 40 ml. 0.3 ml of Ni²⁺-NTA beads (Qiagen) were added to the extract and incubated with agitation for 1 h at 4° C. Beads were poured into a column and washed with 40 ml of wash buffer (50 mM Tris, 20 mM imidazole, 200 mM NaCl). Proteins were eluted in 1 ml of wash buffer supplemented with 200 mM imidazole and immediately re-buffered to 10 mM ammonium carbonate (pH 7.5) using a sephadex G25 column. The purified proteins were analysed by 4-20% SDS-PAGE. Western blots were performed with antibodies against the hexahsitidine tag (Qiagen) and N^{ε}-acetyl-lysine (Santa Cruz).

### Mass Spectrometry

Proteins rebuffered to 10 mM ammonium carbonate (pH 7.5) were mixed 1:1 with 1% formic acid in 50% methanol. Total mass was determined on an LCT time-of-flight mass spectrometer with electrospray ionization icromass). Samples were injected at 10 ml min⁻¹ and calibration performed in positive ion mode using horse heart myoglobin. 60-90 scans were averaged and molecular masses obtained by deconvoluting multiply charged protein mass spectra using MassLynx version 4.1 (Micromass). Theoretical masses of wild-type proteins, were calculated using Protparam (http://us.expasy.org/tools/protparam.html), and theoretical masses for unnatural amino acid containing proteins adjusted manually.

### Example 1

### Selection and Use of the MbPy1RS/MbtRNA_{CUA} Pair

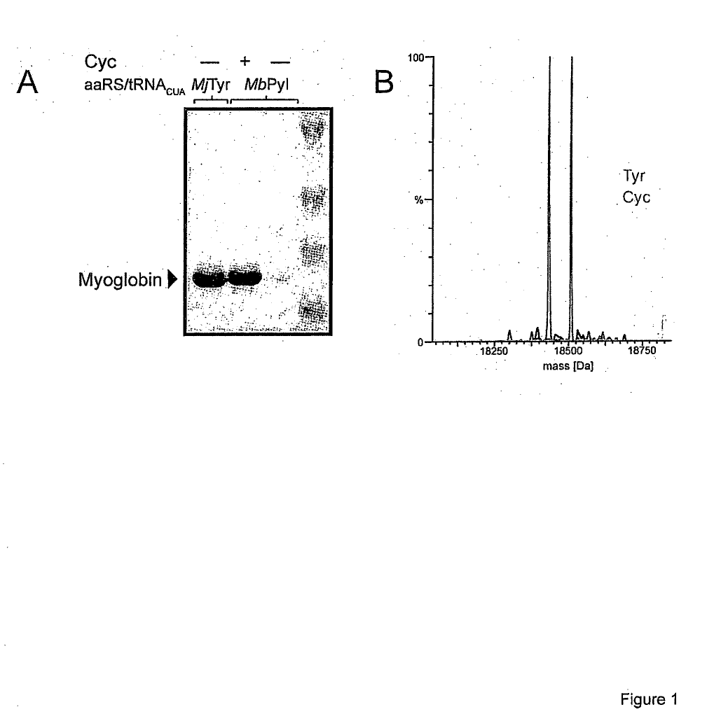

To confirm the activity of the MbPy1RS/MbtRNA_{CUA} pair in E. coli, and the orthogonality of MbtRNA_{CUA} with respect to cellular aminoacyl-tRNA synthetases we examined the ability of the. PyIS and PylT genes, encoding MbPy1RS and MbtRNA_{CUA} respectively to direct the incorporation of the pyrrolysine analog N^{ε}-cyclopentyloxycarbonyl-L-lysine (Cyc, previously demonstrated to be an efficient substrate of MbPy1RS¹⁹) in response to the amber codon. Cells transformed with pBK-Py1S (encoding MbPy1RS) and pREP-Py1T (encoding MbtRNA_{CUA}, an amber mutant of chloramphenicol acetyl transferase, an amber mutant of the T7 RNA polymerase gene, and a green fluorescent protein gene on a T7 promoter) and grown in the presence of Cyc survived on 150 μg ml⁻¹ chloramphenicol and exhibited green fluorescence. When Cyc or pBK-Py1S were withheld from the media cells failed to survive on greater than 20 μg ml⁻¹ chloramphenicol and did not exhibit green fluorescence. These results confirm that MbtRNA_{CUA} is not substantially aminoacylated by endogenous aminoacyl-tRNA synthetases in E. coli and that the MbPy1RS/MbtRNA_{CUA} pair mediates Cyc dependent amber suppression in E. coli. To demonstrate that the MbPy1RS/MbtRNA_{CUA} pair can support protein expression at levels comparable to that of a pair previously used for genetic code expansion we created an expression construct (Myo4TAG-Py1T) containing the genes encoding MbtRNA_{CUA} and sperm whale myoglobin bearing an amber codon in place of the codon for serine 4 and a C-terminal hexahistidine tag. Cells containing Myo4TAG-Py1T, pBK-Py1S and 1 mM Cyc produced full-length myoglobin (FIG. 1), with a purified yield of 2 mg per liter of culture (a comparable yield of myoglobin was obtained when the Methanococcus jannaschii (Mj) tyrosyl-tRNA synthetase tRNA_{CUA} (MjTyrRS/MjtRNA_{CUA}) pair was used to insert tyrosine in response to the amber codon in the same myoglobin gene (FIG. 1). This data. confirms that the MbPy1RS/MbtRNA_{CUA} pair directs amino acid incorporation with a comparable efficiency to a pair previously used for genetic code expansion. Only a trace of full-length myoglobin was detected by Coomassie staining if Cyc, or MbPy1RS were withheld from cells, suggesting that there is a very low level of aminoacylation of MbtRNA_{CUA} by endogenous aminoacyl-tRNA synthetases.

### Example 2

### Quantitative Incorporation

To examine the functional orthogonality of MbPy1RS with respect to cellular tRNAs and to demonstrate that Cyc incorporation with the MbPy1RS/MbtRNA_{CUA} pair is quantitative we acquired electrospray ionization mass spectra of purified myoglobin containing Cyc (FIG. 1). The spectra show a single peak corresponding to the encoded incorporation of Cyc. This data confirms that Cyc is not measurably incorporated in response to sense codons (ie: MbPy1RS is functionally orthogonal) and that natural amino acid are not measurably incorporated in response to the amber codon in the presence of Cyc. Overall, phenotypic data, protein expression data, and mass spectrometry data demonstrate that the MbPy1RS/MbtRNA_{CUA} pair is a highly active, specific and orthogonal pair in E. coli.

### Example 3

### Evolution of N^{ε}-acetyl-lysine Activity

In this example, a method of making a tRNA synthetase capable of binding N^{∈}-acetyl lysine is demonstrated. The method comprises mutating a nucleic acid encoding a parent tRNA synthetase sequence at one or more of L266, L270, Y271, L274 or C313. In this example, each of those residues is mutated. Following mutation, mutants which are capable of binding N^{∈}-acetyl lysine are selected:

### Mutation

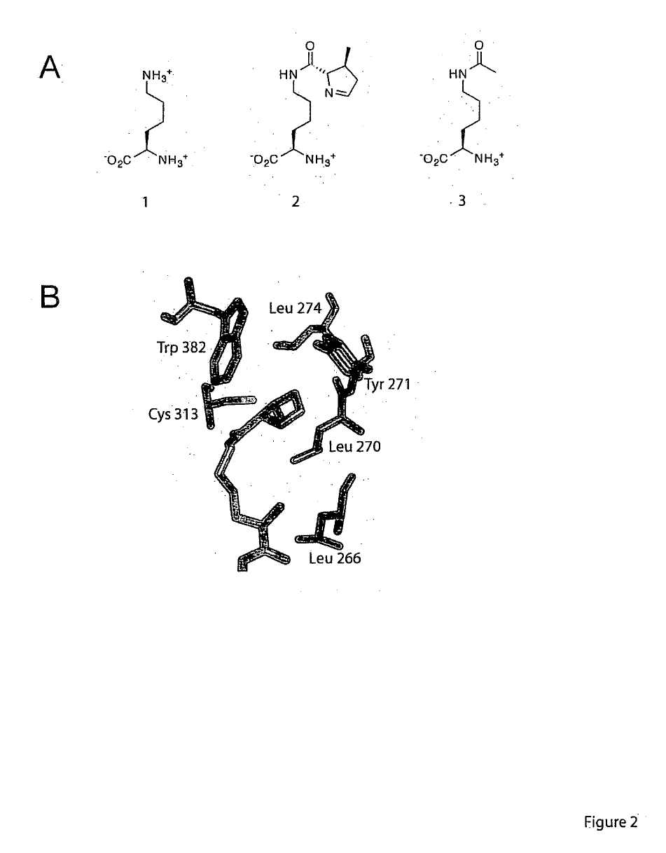

To begin to evolve the MbPy1RS/MbtRNA_{CUA} orthogonal pair for the incorporation of N^{∈}-acetyl-lysine in response to the amber codon we created a library of 10⁸ MbPy1RS mutants in which six residues (Leu 266, Leu 270, Tyr 271, Leu 274, Cys 313, Trp 383) were randomized (FIG. 2). These residues were chosen on the basis of the structure of MbPy1RS in complex with pyrrolysine²³, and are within 6 Å of the bound pyrrole ring of pyrrolysine.

### Selection

To select mutant MbPy1RS/MbtRNA_{CUA} pairs that direct the genetic incorporation of N^{ε}-acetyl-lysine we performed three rounds of selection (positive, negative, positive). In the positive selections cells were transformed with the aminoacyl-tRNA synthetase library and pREP-Py1T and grown in the presence of 1 mM N^{ε}-acetyl-lysine and 50 μg ml⁻¹ chloramphenicol to select active synthetases²². The surviving synthetase clones were subject to a negative selection in the absence of N^{ε}-acetyl-lysine by cotransformation with pBAR Py1T (which contains Py1T and the gene for the toxic ribonuclease barnase in which two codons have been converted to amber codons)²². This step removes aminoacyl-tRNA synthetases that use natural amino acids as substrates.

After three rounds of positive and negative selection the surviving aminoacyl-tRNA synthetase clones were isolated and transformed with pREP-Py1T. Ninety-six clones were screened for N^{ε}-acetyl-lysine dependent chloramphenicol resistance and GFP fluorescence. Twenty-two clones conferred chloramphenicol resistance on E. coli up to 150 μg ml⁻¹ and 20-30 μg ml⁻¹ chloramphenicol in the presence and absence of 2 mM N^{ε}-acetyl-lysine respectively; these clones also showed amino acid dependent GFP fluorescence. The large difference in chloramphenicol resistance in the presence and absence of N^{ε}-acetyl-lysine suggests that the selected synthetases have a substantial in vivo specificity for the insertion of N^{ε}-acetyl-lysine, over all twenty common amino acids found in the cell, in response to the amber codon. Sequencing revealed the twenty-two clones corresponded to two distinct aminoacyl-tRNA synthetase sequences, which we designated AcKRS-1 and AcKRS-2. AcKRS-1 has five mutations (L266V, L270I, Y271F, L274A, C313F) while AcKRS-2 has four mutations (L270I, Y271L, L274A, C313F) with respect to MbPy1RS.

Without wishing to be bound by theory, it is likely that the hydrophobic cavity that binds the pyrrole ring in MbPy1RS is rearranged to bind the acetyl group, and that the difference in volume between the pyrrolysine and N^{ε}-acetyl-lysine is compensated for by the larger volume of the mutant amino acids in the evolved synthetases.

### Example 4

### Method of Making a Polypeptide Comprising N^{∈}-Acetyl Lysine

In this example, polypeptide comprising N^{∈}-acetyl lysine is produced. This is carried out by arranging for the translation of a RNA encoding said polypeptide. This RNA comprises an amber codon.

The translation is carried out in the presence of a polypeptide according to the invention as described in example 3 above, i.e. AcKRS-1 or AcKRS-2. The translation is also carried out in the presence of tRNA capable of being charged with N^{∈}-acetyl lysine, in this example Py1T, and in the presence of N^{∈}-acetyl lysine.

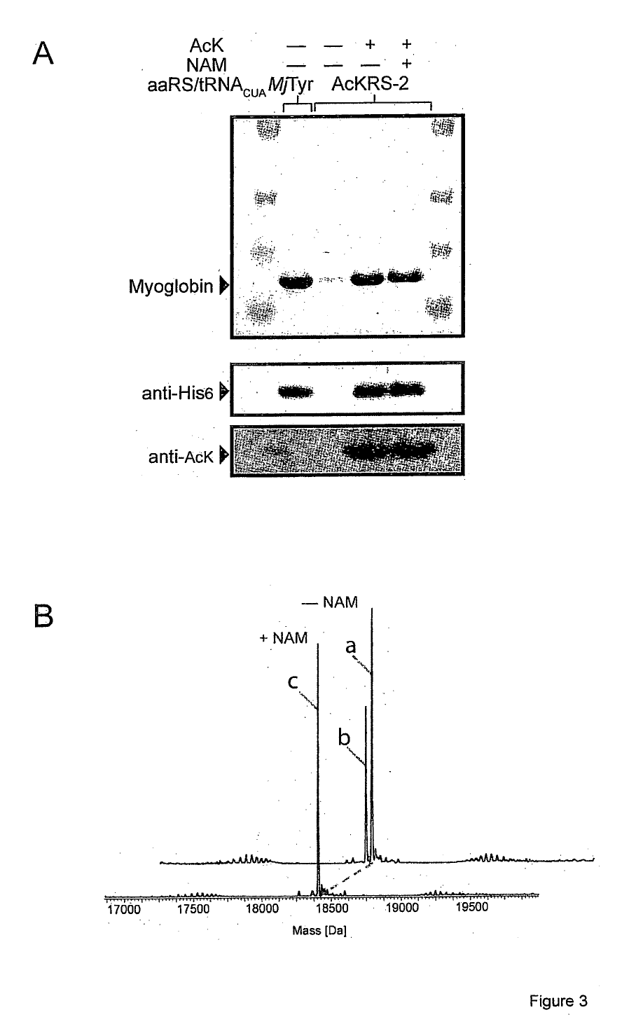

Thus, to demonstrate the fidelity and efficiency of acetyl-lysine incorporation in response to the amber codon, cells containing Myo4TAG-Py1T, AcKRS-1 or AcKRS-2 and 1 mM N^{ε}-acetyl-lysine were used to produce full-length myoglobin. Myoglobin was purified with a yield of 1.5 mg per liter of culture (FIG. 3), which is comparable to yields reported for the incorporation of unnatural amino acids using the most active variants of the MjTyrRS/MjtRNA_{CUA} pair²². Only trace amounts of myoglobin were detected by Coomassie stain or Western blot against C-terminal His-6 tag if N^{ε}-acetyl-lysine was withheld from cells. Western blots against N^{ε}-acetyl-lysine further confirm the incorporation of the amino acid into myoglobin. These data further confirm that the selected aminoacyl-tRNA synthetases are very selective for N^{ε}-acetyl-lysine.

### Example 5

### Method of Making a Polypeptide Comprising N^{∈}-Acetyl Lysine

In this example, the polypeptide is produced under the inhibition of deacetylase. Polypeptide is first produced according to example 4. Electrospray ionization mass spectroscopy of myoglobin purified, from cells containing AcKRS-2, Myo4TAG-Py1T and N^{ε}-acetyl-lysine gave two peaks (FIG. 3): one peak corresponds to the incorporation of N^{ε}-acetyl-lysine, while the second peak has a mass of 42 Da less. We assigned the second peak to myoglobin bearing lysine in place of N^{ε}-acetyl-lysine. We reasoned that since myoglobin expression from Myo4TAG-Py1T is dependent on the addition of N^{ε}-acetyl-lysine to cells, the lysine containing myoglobin must be derived from post-translational de-acetylation in E. coli.

E. coli has a single characterized de-acetylase, CobB: a sirtuin family, nicotinamide adenine dinucleotide dependent enzyme^{24,25}. Since the sirtuin family of enzymes are known to be potently inhibited by nicotinamide (NAM) we performed protein expression according to example 4, but in the additional presence of this inhibitor. Electrospray ionization spectra of myoglobin produced from cells containing nicotinamide (FIG. 3) gave a single peak corresponding to the acetylated protein, with no peak observed for deacetylated protein. We conclude that nicotinamide completely inhibits the post-translational de-acetylation of genetically incorporated acetyl-lysine in E. coli.

### Summary of Examples Section

In conclusion, we have confirmed the orthogonality of MbtRNA_{CUA} with respect to cellular aminoacyl-tRNA synthetases in E. coli, demonstrated the orthogonality of MbPy1RS with respect to cellular tRNAs in E. coli and demonstrated the efficiency of this orthogonal pair in E. coli. We have evolved the MbPy1RS/MbtRNA_{CUA} orthogonal pair to direct the incorporation of N^{ε}-acetyl-lysine, with high translational fidelity and efficiency, into proteins expressed in E. coli. Furthermore we have developed an inhibitor based strategy to eradicate the initially observed post-translational deacetylation of co-translationally incorporated N^{ε}-acetyl-lysine in E. coli. Thus the materials and techniques described here are useful for producing site-specifically acetylated recombinant proteins.

### REFERENCES

1. Shogren-Knaak, M. et al. Histone H4-K16 acetylation controls chromatin structure and protein interactions. Science 311, 844-847 (2006).
2. Jenuwein, T. & Allis, C. D. Translating the histone code. Science 293, 1074-1080 (2001).
3. Kouzarides, T. Chromatin modifications and their function: Cell 128, 693-705 (2007).
4. Gu, W. & Roeder, R. G. Activation of p53 sequence-specific DNA binding by acetylation of the p53 C-terminal domain. Cell 90, 595-606 (1997).
5. Groth, A., Rocha, W., Verreault, A. & Almouzni, G. Chromatin challenges during DNA replication and repair. Cell 128, 721-733 (2007).
6. Peterson, C. L. & Cote, J. Cellular machineries for chromosomal DNA repair. Genes Dev 18, 602-616 (2004).
7. Hubbert, C. et al. HDAC6 is a microtubule-associated deacetylase. Nature 417, 455-458 (2002).
8. Serrador, J. M. et al. HDAC6 deacetylase activity links the tubulin cytoskeleton with immune synapse organization. Immunity 20, 417-428 (2004).
9. Yang, X. J. & Gregoire, S. Metabolism, cytoskeleton and cellular signalling in the grip of protein Nepsilon—and O-acetylation. EMBO Rep 8, 556-562 (2007).
10. Scroggins, B. T. et al. An acetylation site in the middle domain of Hsp90 regulates chaperone function. Mol Cell 25, 151-159 (2007).
11. Bannister, A. J., Miska, E. A., Gorlich, D. & Kouzarides, T. Acetylation of importin-alpha nuclear import factors by CBP/p300. Curr Biol 10, 467-470 (2000).
12. Yuan, Z. L., Guan, Y. J., Chatterjee, D. & Chin, Y. E. Stat3 dimerization regulated by reversible acetylation of a single lysine residue. Science 307, 269-273 (2005).
13. Cohen, H. Y. et al. Acetylation of the C terminus of Ku70 by CBP and PCAF controls Bax-mediated apoptosis. Mol Cell 13, 627-638 (2004).
14. Kouzarides, T. Acetylation: a regulatory modification to rival phosphorylation? Embo J 19, 1176-1179 (2000).
15. Hao, B. et al. A new UAG-encoded residue in the structure of a methanogen methyltransferase. Science 296, 1462-1466 (2002).
16. Srinivasan, G., James, C. M. & Krzycki, J. A. Pyrrolyside encoded by UAG in Archaea: charging of a UAG-decoding specialized tRNA. Science 296, 1459-1462 (2002).
17. Blight, S. K. et al. Direct charging of tRNA(CUA) with pyrrolysine in vitro and in vivo. Nature 431, 333-335 (2004).
18. Ambrogelly, A. et al. Pyrrolysine is not hardwired for cotranslational insertion at UAG codons. Proc Natl Acad Sci USA 104, 3141-3146 (2007).
19. Polycarpo, C. R. et al. Pyrrolysine analogues as substrates for pyrrolysyl-tRNA synthetase. FEBS Lett 580, 6695-6700 (2006).
20. Kim, S. C. et al. Substrate and functional diversity of lysine acetylation revealed by a proteomics survey. Mol Cell 23, 607-618 (2006).
21. Allfrey, V. G., Faulkner, R. & Mirsky, A. E. Acetylation and Methylation of Histones and Their Possible Role in the Regulation of Rna Synthesis. Proc Natl Acad Sci USA 51, 786-794 (1964).
22. Chin, J. W., Martin, A. B., King, D. S., Wang, L. & Schultz, P. G. Addition of a photocrosslinking amino acid to the genetic code of Escherichiacoli. Proc Natl Acad Sci USA 99, 11020-11024 (2002).
23. Kavran, J. M. et al. Structure of pyrrolysyl-tRNA synthetase, an archaeal enzyme for genetic code innovation. Proc Natl Acad Sci USA 104, 11268-11273 (2007).
24. Blander, G. & Guarente, L. The Sir2 family of protein deacetylases. Annu Rev Biochem 73, 417-435 (2004).
25. Schwer, B., Bunkenborg, J., Verdin, R. O., Andersen, J. S. & Verdin, E. Reversible lysine acetylation controls the activity of the mitochondrial enzyme acetyl-CoA synthetase 2. Proc Nall Acad Sci USA 103, 10224-10229 (2006).
26. Luger, K., Mader, A. W., Richmond, R. K., Sargent, D. F. & Richmond, T. J. Crystal structure of the nucleosome core particle at 2.8 A resolution. Nature 389, 251-260 (1997).
27. Wang, K., Neumann, H., Peak-Chew, S. Y. & Chin, J. W. Evolved orthogonal ribosomes enhance the efficiency of synthetic genetic code expansion. Nat Biotechnol 25, 770-777 (2007).
28. Santoro, S. W., Wang, L., Herberich, B., King, D. S. & Schultz, P. G. An efficient system for the evolution of aminoacyl-tRNA synthetase specificity. Nat Biotechnol 20, 1044-1048 (2002).
29. Rackham, O. & Chin, J. W. A network of orthogonal ribosome x mRNA pairs. Nat Chem Biol 1, 159-166 (2005).
30. Christ, D., Famm, K. & Winter, G. Tapping diversity lost in transformations—in vitro amplification of ligation reactions. Nucleic Acids Res 34, e108 (2006).

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A tRNA synthetase capable of binding N^{∈}-acetyl lysine.

2. A tRNA synthetase according to claim 1 wherein said synthetase comprises a polypeptide having at least 90% sequence identity to the amino acid sequence of MbPy1 RS.

3. A tRNA synthetase according to claim 2 wherein said tRNA synthetase comprises amino acid sequence corresponding to the amino acid sequence of at least L266 to C313 of MbPy1RS, or a sequence having at least 90% identity thereto.

4. A tRNA synthetase according to claim 3 wherein said polypeptide comprises a mutation relative to the wild type MbPy1RS sequence at one or more of L266, L270, Y271, L274 or C313.

5. A tRNA synthetase according to claim 4 wherein said at least one mutation is at L270, Y271, L274 or C313.

6. A tRNA synthetase according to claim 5 wherein said at least one mutation is at L270, L274 or C313.

7. A tRNA synthetase according to claim 1 which comprises Y271L.

8. A tRNA synthetase according to claim 1 which comprises Y271F.

9. A tRNA synthetase according to claim 1 which comprises L266V.

10. A tRNA synthetase according to claim 1 which comprises L270I, Y271L, L274A, and C313F.

11. A tRNA synthetase according to claim 1 which comprises L266V, L2701, Y271F, L274A, and C313F.

12. A nucleic acid comprising nucleotide sequence encoding a polypeptide according to claim 1.

13. (canceled)

14. A method according to claim 15 wherein said tRNA comprises MbtRNA_{CUA}.

15. A method of making a polypeptide comprising N^{∈}-acetyl lysine comprising arranging for the translation of a RNA encoding said polypeptide, wherein said RNA comprises an amber codon, wherein said translation is carried out in the presence of a polypeptide according to claim 1 and in the presence of tRNA capable of being charged with N^{∈}-acetyl lysine, and in the presence of N^{∈}-acetyl lysine.

16. A method according to claim 15 wherein said translation is carried out in the presence of an inhibitor of deacetylation.

17. A method according to claim 16 wherein said inhibitor comprises nicotinamide (NAM).

18. A method of making a polypeptide comprising N^{∈}-acetyl lysine, said method comprising modifying a nucleic acid encoding said polypeptide to provide an amber codon at one or more position(s) corresponding to the position(s) in said polypeptide where it is desired to incorporate N^{∈}-acetyl lysine.

19. A method according to claim 18 wherein modifying said nucleic acid comprises mutating a codon for lysine to an amber codon (TAG).

20. A homogeneous recombinant protein comprising N^{∈}-acetyl lysine prepared according to a method of claim 15.

21. (canceled)

22. A vector comprising nucleic acid according to claim 12.

23. A vector according to claim 22, said vector further comprising nucleic acid sequence encoding a tRNA substrate of said tRNA synthetase.

24. A vector according to claim 23 wherein said tRNA substrate is encoded by the MbPyIT gene.

25. A cell comprising a nucleic acid according to claim 12.

26. A cell according to claim 25 which further comprises an inactivated de-acetylase gene.

27. A cell according to claim 26 wherein said deactivated de-acetylase gene comprises a deletion or disruption of CobB.

28. A kit comprising a vector according to claim 22, and an amount of nicotinamide.

29. A method of making a tRNA synthetase capable of binding N^{∈}-acetyl lysine, said method comprising mutating a nucleic acid encoding a parent tRNA synthetase sequence at one or more of L266, L270, Y271, L274 or C313, and selecting one or more mutants which are capable of binding N^{∈}-acetyl lysine.

30. (canceled)

31. A cell comprising a vector according to claim 22.

32. A kit comprising a cell according to claim 25, and an amount of nicotinamide.


SEQUENCE LISTING (24 sequences)

SEQ ID NO: 1 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMXAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 2 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTXYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 3 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLXNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 4 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLYNYXRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 5 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLYNYLRKLDRILPDPIKIFEVGPCYRKESDGKEHLEEFTMVNFXQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 6 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLLNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 7 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLFNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 8 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMVAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 9 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTILNYARKLDRILPDPIKIFEVGPCYRKESDGKEHLEEFTMVNFFQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 10 - protein, 419 aa, Artificial
  mbPylRS with mutation(s)
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMVAPTIFNYARKLDRILPDPIKIFEVGPCYRKESDGKEHLEEFTMVNFFQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 11 - DNA, 35 bp, Artificial
  primer
caagcggccgcgaattcagcgttacaagtattaca

SEQ ID NO: 12 - DNA, 27 bp, Artificial
  primer
gaccactgcagatccttagcgaaagct

SEQ ID NO: 13 - DNA, 32 bp, Artificial
  primer
gcgacgctgcagtggcggaaaccccgggaatc

SEQ ID NO: 14 - DNA, 39 bp, Artificial
  primer
ggaaaccgcgcggccgcgggaacctgatcatgtagatcg

SEQ ID NO: 15 - DNA, 35 bp, Artificial
  primer
ctagatctatgactagtatccttagcgaaagctaa

SEQ ID NO: 16 - DNA, 35 bp, Artificial
  primer
atactagtcatagatctagcgttacaagtattaca

SEQ ID NO: 17 - DNA, 55 bp, Artificial
  primer
gctagatctgggaacctgatcatgtagatcgaatggactctaaatccgttcagcc

SEQ ID NO: 18 - DNA, 55 bp, Artificial
  primer
gatactagttggcggaaaccccgggaatctaacccggctgaacggatttagagtc

SEQ ID NO: 19 - DNA, 56 bp, Artificial
  primer
gcgcaggaaaggtctcaaactttnnkcaaatgggcagcggctgcacccgtgaaaac

SEQ ID NO: 20 - DNA, 52 bp, Artificial
  primer
gcgcagagtaggtctcaagttaaccatggtgaattcttccaggtgttctttg

SEQ ID NO: 21 - DNA, 73 bp, Artificial
  primer
gcgcaggtctcaccgatgnnkgccccgaccnnknnkaactatnnkcgtaaactggatcgtattctgccgggtc

SEQ ID NO: 22 - DNA, 57 bp, Artificial
  primer
gcgcagagtaggtctcatcggacgcaggcacaggtttttatccacgcggaaaatttg

SEQ ID NO: 23 - DNA, 59 bp, Artificial
  primer
gcgcaggaaaggtctcaaaaccgnnkattggcgcgggttttggcctggaacgtctgctg

SEQ ID NO: 24 - DNA, 56 bp, Artificial
  primer
gcgcagagtaggtctcagtttatcaatgccccattcacgatccaggctaaccggac